(19) United States
(12) Patent Application Publication
(10) Pub. No.: US 2019/0105240 A1
(43) Pub. Date: Apr. 11, 2019

(54) ANTIPERSPIRANT STICK IN THE FORM OF AN O/W EMULSION

(71) Applicant: BEIERSDORF AG, Hamburg (DE)

(72) Inventors: Ilka OELRICHS, Tornesch (DE); Franziska SCHECKER, Buchholz (DE); Marita HALLMANN, Hamburg (DE)

(21) Appl. No.: 16/087,127

(22) PCT Filed: Mar. 9, 2017
(86) PCT No.: PCT/EP2017/055562

§ 371 Date: Sep. 21, 2018

(30) Foreign Application Priority Data
Mar. 22, 2016 (DE) 10 2016 204 685.3

(51) Int. Cl. A61K 8/02; A61K 8/06; A61K 8/34; A61K 8/39; A61K 8/36; A61K 8/92; A61K 8/368; A61Q 15/00

(52) U.S. Cl. CPC A61K 8/0229; A61K 8/062; A61K 8/345; A61K 8/342; A61Q 15/00; A61K 8/361; A61K 8/92; A61K 8/368; A61K 8/39

(57) ABSTRACT

The invention relates to a cosmetic preparation in the form of an O/W emulsion with improved skin compatibility, comprising short-chain alkane -1,2-dioles, emulsifiers, fatty alcohols, oils, waxes, carboxylic acids, antiperspirant agent and water, the preparation being characterised in that it is solid and dimensionally stable.

The present invention relates to a cosmetic preparation in the form of an O/W emulsion having improved skin compatibility, comprising short-chain alkane-1,2-diols, emulsifiers, fatty alcohols, oils, waxes, carboxylic acids, antiperspirant active ingredient and water, the preparation being characterized in that it is solid and dimensionally stable.

Antiperspirants (APs) or deodorants (DOs) serve to eliminate or prevent body odor, which arises when inherently odorless fresh sweat is decomposed by microorganisms.

In general language usage, there is not always a clear separation of the terms “deodorant” and “antiperspirant”. On the contrary—especially also in the German-speaking world—products for underarm use are, as a whole, referred to as deodorants. It is immaterial whether there is also an antiperspirant action.

Antiperspirants (APs) are agents which are intended to impede the secretion of sweat (perspiration) by blocking the openings of sweat glands. In the vast majority of antiperspirants, astringents—predominantly aluminum salts such as aluminum hydroxychloride (aluminum chlorohydrate, ACH), aluminum chloride or complexes of aluminum zirconium tetrachlorohydrate and glycine—can reduce sweating. Besides an antiperspirant or sweat-reducing action, aluminum compounds also exhibit an antimicrobial action and are thus also deodorants.

For skin application, active ingredients with antiperspirant action are incorporated into cosmetic preparations. One type of antiperspirants for underarm application that is popular with the consumer are solid cosmetic preparations with antiperspirant action, which are frequently referred to as antiperspirant sticks. During the application of antiperspirant sticks, the solid preparation is transferred to the skin by abrasion.

Nowadays, antiperspirants are body-care products that are part of daily life. In many cases, the consumer applies them not only in the morning, but also after sport activities with subsequent shower. It is therefore common practice to carry along antiperspirants every day as personal belongings. This means that the products are sometimes exposed to extreme temperatures. Heating due to solar radiation to temperatures around 50° C. is possible in the summer in southern and central Europe and also throughout the year in equatorial regions.

By contrast, in the winter in central Europe and in colder regions of the world, the products are exposed to temperatures below freezing. Furthermore, the products can, especially in the summer, be exposed to strong temperature fluctuations between air-conditioned buildings and outdoor air. Under these conditions, there are often deformations or freezing materials with subsequent cracking, which are described as unaesthetic by the consumer. The disadvantage here for the manufacturer is that the resale rate of the products drops.

WO 9959537 A1 discloses cosmetic sticks which already melt from a temperature of 30° C. and thus have inadequate dimensional stability.

Furthermore, DE 19962878 A1 discloses aqueous antiperspirant compositions which have a water content of up to 70% by weight. An advantage of the high water content of the antiperspirant compositions is that a fresh, cooling sensation is perceived when applying the composition to the skin. However, a disadvantage of the antiperspirant compositions is that the antiperspirant compositions preferably have a viscosity of 200 000-1 500 000 mPa·s at 21° C. Thus, the antiperspirant compositions can be referred to as creams. A disadvantage of creams is that they are not solid and dimensionally stable and are thus in most cases applied under the armpits by hand or with an aid, such as a cloth or a pad. By contrast, use of solid antiperspirant sticks is more practical and preferred by the consumer, since they can be applied directly from the packaging.

WO 2006012971 A1 discloses cosmetic preparations with antiperspirant action that are based on an O/W emulsion, which cosmetic preparations contain short-chain alkane-1,2-dials with an alkyl chain having from 3 to 5 carbon atoms in a proportion of from 4% by weight to 25% by weight, based on the total weight of the preparation. Solid cosmetic formulations are only obtained when the proportion by weight of alkane-1,2-diols with an alkyl chain having from 3 to 5 carbon atoms is at least 12% by weight, based on the total weight of the preparation.

The incorporation of alkane-1,2-diols with an alkyl chain having from 3 to 5 carbon atoms is a customary method for lowering the freezing point and thus preventing freezing of antiperspirant sticks formulated as oil-in-water emulsion (O/W emulsion).

However, a disadvantage is that alkane-1,2-diols, such as propane-1,2-diol for example, may have a skin-irritating action. For instance, propane-1,2-diol has a strong skin-irritating action when used in of concentrations above 50% by weight, based on the total weight of the preparation (A. Scheman, S. Jacob, M. Zirwas et al., Dis. Mon. 54, 7-156 (2008)). Although the skin-irritating action of propane-1,2-diol decreases with decreasing concentration, there are known studies which were able to demonstrate allergic skin reactions in 4% of the participants in epicutaneous tests with 10% by weight of propane-1,2-diol (Propylene Glycol Information Update, American Chemistry Council's Propylene Oxide/Propylene Glycol Panel, 2001). Furthermore, the publication Antitranspirant and Deodorant Allergy—Diagnosis and Management (M. J. Zirwas, J. Moennich, Journal of clinical and Aesthetic Dermatology, Vol. 1, No. 3 (2008)) indicates that propane-1,2-diol is one of the most frequently occurring allergens in antiperspirants and deodorants. Even in the case of propane-1,2-diol concentrations of from 3% by weight to 5% by weight, it is not possible to rule out an allergic reaction of users with a propane-1,2-diol allergy.

It is an object of the present invention to provide a cosmetic preparation with antiperspirant action in the form of an O/W emulsion that does not have the disadvantages of the prior art. In particular, the cosmetic preparation should be solid and dimensionally stable and have, at the same time, an improved skin compatibility and/or a low allergenic potential.

Surprisingly, the above objects are achieved by the present invention.

The present invention provides a cosmetic preparation in the form of an O/W emulsion, comprising
a) from 0.1% by weight to 2.5% by weight of one or more alkane-1,2-diols with an alkyl chain having from 3 to 5 C atoms,
b) two or more emulsifiers, containing at least one O/W emulsifier having an HLB value greater than 8 and at least one W/O emulsifier having an HLB value up to a maximum of 8,
c) one or more fatty alcohols selected from aliphatic, linear, primary alkanols having a chain length of from 6 to 22 C atoms,
d) one or more oils liquid at 20° C. and 1013 hPa,
e) one or more waxes which melt from a temperature of 40° C. at 1013 hPa without decomposition,
f) one or more saturated and/or unsaturated linear carboxylic acids having a chain length of from 10 to 20 C atoms,
g) one or more active ingredients with antiperspirant action, and
h) water,

based in each case on the total weight of the preparation, the cosmetic preparation having a penetration force value of from 300 gram-force to 600 gram-force.

The cosmetic preparation with antiperspirant action of the present invention has not only an improved skin compatibility and/or a low allergenic potential, but is, at the same time, solid and dimensionally stable.

The invention also provides a method for decreasing or inhibiting perspiration, the method comprising the application to the skin of a quantity of the cosmetic preparation according to the invention that is effective against perspiration.

The invention also provides for the use of the cosmetic preparation according to the invention on the skin for decreasing perspiration.

Since water forms the outer phase in the preparation according to the invention, residues remaining on the skin after application can be easily rinsed off with water. Residues on clothing can likewise be easily removed with water.

Unless otherwise specified, all specified percentages by weight (% by weight) below are based on the total weight of the preparation according to the invention.

The cosmetic preparation according to the invention contains one or more alkane-1,2-diols with an alkyl chain having from 3 to 5 C atoms in a total proportion of from 0.1% by weight to 2.5% by weight, preferably from 0.2% by weight to 2% by weight.

According to the invention, it is preferred when the cosmetic preparation according to the invention contains propane-1,2-diol as alkane-1,2-diol with an alkyl chain having from 3 to 5

C atoms.

In particular, it is preferred when, besides propane-1,2-diol, the cosmetic preparation according to the invention does not contain any further alkane-1,2-diols with an alkyl chain having from 3 to 5 C atoms.

Since the one or more alkane-1,2-diols are most probably situated in the aqueous phase of the O/W emulsion, an incorporation into the emulsion is associated with a lowering of the freezing point. However, since alkane-1,2-diols are also solvents, it is expected by a person skilled in the art that, upon addition, the dimensional stability of the emulsion will deteriorate, especially at relatively high temperatures, and a creamy solution will be obtained. It was found that, surprisingly, the cosmetic preparation according to the invention is dimensionally stable.

In general, emulsifiers lower the interfacial tension between oil and water phases and achieve not only the decrease in the interfacial work, but also a stabilization of the emulsion formed. They stabilize the emulsion formed by means of interfacial films and by formation of steric or electric barriers, the result being that the merging (coalescence) of the emulsified particles is prevented.

For compounds to be able to be effective as emulsifiers, they must have a certain molecular structure. A structural characteristic of such compounds is their amphiphilic molecular structure. The molecule of such a compound has at least one group with affinity for substances of strong polarity (polar group) and at least one group with affinity for nonpolar substances (apolar group).

A distinction is made here between nonionic, anionic and cationic emulsifiers. One characteristic of the hydrophilicity of a given emulsifier is its HLB value, which is yielded by the following formula: HLB=20×(1−M_{lipophilic}/M), where M_{lipophilic} stands for the molar mass of the lipophilic portion in the emulsifier and M stands for the molar mass of the entire emulsifier.

In general, emulsifiers having an HLB value up to a maximum of 8 are considered to be W/O emulsifiers. By contrast, O/W emulsifiers have HLB values of greater than 8.

The preparation according to the invention comprises two or more emulsifiers, containing at least one O/W emulsifier having an HLB value greater than 8 and at least one W/O emulsifier having an HLB value up to a maximum of 8.

Advantageously, the total proportion of emulsifiers is from 1.5% by weight to 5% by weight, preferably from 2% by weight to 3% by weight.

The emulsifiers according to the invention can be selected from the group of anionic, cationic, amphoteric and nonionic emulsifiers. The use of nonionic emulsifiers is preferred according to the invention.

Nonionic emulsifiers according to the invention are, for example, propylene glycol isostearate (HLB 2.5), glycol stearate (HLB 2.9), glyceryl isostearate (HLB 3.5), sorbitan sesquioleate (HLB 3.7), glyceryl stearate (HLB 3.8), lecithin (HLB 4), sorbitan oleate (HLB 4.3), sorbitan monostearate NF (HLB 4.7), sorbitan stearate (HLB 4.7), sorbitan isostearate (HLB 4.7), steareth-2 (HLB 4.9), oleth-2 (HLB 4.9), glyceryl laurate (HLB 5.2), ceteth-2 (HLB 5.3), PEG-30 dipolyhydroxystearate (HLB 5.5), glyceryl stearate SE (HLB 5.8), sorbitan stearate (and) sucrose cocoate (HLB 6), PEG-4 dilaurate (HLB 6), PEG-8 dioleate (HLB 8), sorbitan laurate (HLB 8.6), PEG-40 sorbitan peroleate (HLB 9), laureth-4 (HLB 9.7), PEG-7 glyceryl cocoate (HLB 10), PEG-20 almond glycerides (HLB 10), PEG-25 hydrogenated castor oil (HLB 10.8), stearamide MEA (HLB 11), glyceryl stearate (and) PEG-100 stearate (HLB 11), polysorbate 85 (HLB 11), PEG-7 olivate (HLB 11), cetearyl glucoside (HLB 11), PEG-8 oleate (HLB 11.6), polyglyceryl-3 methylglucose distearate (HLB 12), PEG-10 stearate (HLB 12), oleth-10 (HLB 12.4), oleth-10/polyoxyl 10 oleyl ether NF (HLB 12.4), ceteth-10 (HLB 12.9), PEG-8 laurate (HLB 13), ceteareth-12 (HLB 13.5), cocamide MEA (HLB 13.5), polysorbate 60 NF (HLB 14.9), polysorbate 60 (HLB 14.9), PEG-40 hydrogenated castor oil (HLB 15), polysorbate 80 (HLB 15), isosteareth-20 (HLB 15), PEG-60 almond glycerides (HLB 15), polysorbate 80 NF (HLB 15), PEG-150 laurate, PEG-20 methyl glucose sesquistearate (HLB 15), ceteareth-20 (HLB 15.2), oleth-20 (HLB 15.3), steareth-20 (HLB 15.3), steareth-21 (HLB 15.5), ceteth-20 (HLB 15.7), isoceteth-20 (HLB 15.7), PEG-30 glyceryl laurate (HLB 16), polysorbate 20 (HLB 16.7), polysorbate 20 NF (HLB 16.7), laureth-23 (HLB 16.9), PEG-100 stearate (HLB 18.8), steareth-100 (HLB 18.8), PEG-80 sorbitan laurate (HLB 19.1).

Nonionic 01W emulsifiers preferred according to the invention have an HLB value greater than 8, particularly preferably an HLB value between 13 and 18, very particularly preferably an HLB value between 14.5 to 16.5.

In a further embodiment according to the invention, preference is given to selecting nonionic O/W emulsifiers which are addition products of from 15 to 25 ethylene oxide units onto saturated or unsaturated or partially saturated alkanols having a chain length of from 12 to 20 C atoms, preference being given in particular to addition products of from 20 to 21 ethylene oxide units onto saturated or unsaturated or partially saturated alkanols having a chain length of 18 C atoms.

In a further particularly preferred embodiment according to the invention, nonionic O/W emulsifiers are selected which have an HLB value between 14.5 and 16.5 and are addition products of from 20 to 21 ethylene oxide units onto saturated or unsaturated or partially saturated alkanols having a chain length of 18 C atoms.

Nonionic W/O emulsifiers preferred according to the invention have an HLB value up to a maximum of 8, particularly preferably an HLB value between 3 and 7, very particularly preferably an HLB value between 4 and 6.

In a further embodiment according to the invention, preference is given to selecting nonionic W/O emulsifiers which are addition products of from 1 to 4 ethylene oxide units onto saturated or unsaturated or partially saturated alkanols having a chain length of from 12 to 20 C atoms, preference being given in particular to addition products of from 2 to 3 ethylene oxide units onto saturated or unsaturated or partially saturated alkanols having a chain length of 18 C atoms.

In a further particularly preferred embodiment according to the invention, nonionic W/O emulsifiers are selected which have an HLB value between 14.5 and 16.5 and are addition products of from 2 to 3 ethylene oxide units onto saturated or unsaturated or partially saturated alkanols having a chain length of 18 C atoms.

Preference is given to the use of two nonionic O/W emulsifiers and of one nonionic W/O emulsifier.

Furthermore, it is preferred when the respective emulsifiers are individually present in a proportion of from 0.5% by weight to 1% by weight.

Mixtures according to the invention composed of nonionic W/O and O/W emulsifiers can be preferably selected from steareth-2 as W/O emulsifier and oleth-20 and steareth-21 as O/W emulsifiers.

It is especially advantageous in the context of the invention when no further emulsifiers are present besides steareth-2, oleth-20 and steareth-21.

Furthermore, it is according to the invention that one or more fatty alcohols are present in the cosmetic preparation. The fatty alcohols below are, in the context of this invention, certainly not referred to as emulsifiers, oils or waxes. Fatty alcohols in the context of this invention are exclusively aliphatic, linear, primary alkanols having a chain length of from 6 to 22 C atoms.

Fatty alcohols according to the invention are preferably selected from the group consisting of lauryl alcohol, myristyl alcohol, cetyl alcohol, stearyl alcohol, margaryl alcohol, arachidyl alcohol and behenyl alcohol.

Particular preference is given to selecting therefrom cetyl alcohol, stearyl alcohol and/or mixtures thereof. Mixtures of cetyl alcohol and stearyl alcohol are known under the trivial name cetyl stearyl alcohol. Such a mixture can be purchased, inter alia, under the brand name Kolliwax CSA50 from BASF or under the name Nafol 1618-JA from Sasol.

It is especially advantageous in the context of the present invention when no further fatty alcohols are present in the cosmetic preparation according to the invention besides cetyl alkohol, stearyl alcohol and/or cetyl stearyl alcohol.

The fatty alcohols according to the invention are advantageously present in the cosmetic preparation according to the invention in a total proportion of from 0.5% by weight to 5% by weight, preferably from 0.75% by weight to 3.1% by weight.

Furthermore, the cosmetic preparation according to the invention contains one or more oils liquid at 20° C. and 1013 hPa.

The below-listed substance groups and chemicals within the properties specified are, in the context of this invention, to be referred to exclusively as oil. They are certainly not to be referred to as waxes or emulsifiers.

Perfume oils and essential oils are, in the context of the present invention, certainly not to be referred to as oil. Instead, perfume oils and essential oils are, in the context of the present invention, referred to as perfume.

It is advantageous in the context of the present invention when the proportion of the one or more oils liquid at 20° C. and 1013 hPa in the cosmetic preparation according to the invention is from 18% by weight to 27% by weight, preferably from 20% by weight to 25% by weight, especially preferably from 21% by weight to 24% by weight.

It has been found that, surprisingly, a cosmetic preparation according to the invention containing one or more oils liquid at 20° C. and 1013 hPa in a proportion of from 21% by weight to 24% by weight leaves the user with a pleasant, smooth sensation when used as antiperspirant stick on the skin.

The oils according to the invention are selected from the addition products of 6-60 propylene oxide units onto monohydric or polyhydric alkanols having a chain length of from 3 to 22 carbon atoms. In the context of the invention, these are referred to as ether oils. Inventive examples of said ether oils are PPG-9 butyl ether, PPG-12 butyl ether, PPG-14 butyl ether,

PPG-15 butyl ether, PPG-16 butyl ether, PPG-17 butyl ether, PPG-18 butyl ether, PPG-2 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether and PPG-15 stearyl ether, provided that they are liquid at 20° C. and 1013 hPa.

Further oils according to the invention can be selected from the group of esters made from saturated and/or unsaturated, branched and/or unbranched alkane carboxylic acids of a chain length of from 3 to 17 C atoms and unsaturated and/or unsaturated, branched and/or unbranched alcohols of a chain length of from 3 to 10 C atoms and also from the group of esters made from aromatic carboxylic acids and unsaturated and/or unsaturated, branched and/or unbranched alcohols of a chain length of from 3 to 15 C atoms, provided that the ester oil selected is liquid at 20° C. Such ester oils can be advantageously selected from the group consisting of isopropyl myristate, isopropyl palmitate, isopropyl stearate, isopropyl oleate, 2-ethylhexyl palmitate, 2-ethylhexyl laurate, C12-15 alkyl benzoate, and also synthetic, semisynthetic and natural mixtures of such esters, provided that the ester oil selected is liquid at 20° C. and 1013 hPa.

According to the invention, preference is given to using a mixture of three oils, wherein two oils are selected from the group of ether oils and one oil is selected from the group of ester oils, and all the oils used are liquid at 20° C. and 1013 hPa.

According to the invention, the weight ratio of the ether oils to the ester oil in the cosmetic preparation is from 4:1 to 1:1, preferably from 3:1 to 2:1.

In one advantageous embodiment according to the invention, the oil mixtures comprise two ether oils and one ester oil, the ester oil present being C12-15 alkyl benzoate.

In a further advantageous embodiment according to the invention, the oil mixtures comprise two ether oils and one ester oil, the ether oils present being PPG-14 butyl ether and PPG-15 stearyl ether.

In a particularly advantageous embodiment according to the invention, the oil mixtures comprise two ether oils and one ester oil, the ether oils selected being PPG-14 butyl ether and PPG-15 stearyl ether and the ester oil selected being C12-15 alkyl benzoate.

In a very particularly advantageous embodiment, the oil mixture comprises from 22% by weight to 28% by weight of PPG-14 butyl ether, from 40% by weight to 51% by weight of PPG-15 stearyl ether and from 23% by weight to 33% by weight of C12-15 alkyl benzoate, based on the total weight of the oil mixture.

It is especially advantageous in the context of the invention when, besides PPG-14 butyl ether, PPG-15 stearyl ether and C12-15 alkyl benzoate, no further oils liquid at 20° C. and 1013 hPa are present in the preparation according to the invention.

The cosmetic preparation according to the invention comprises one or more waxes which melt from a temperature of 40° C. at 1013 hPa without decomposition.

Waxes according to the invention are plant and/or animal waxes, such as, for example, carnauba wax (Copernicia Cerifera Cera), rice wax, candelilla wax, sunflower wax, beeswax, spermaceti, cork wax, montan wax and cotton wax.

Further waxes according to the invention are selected from the group of esters which are produced by means of esterification of a linear or branched, saturated, partially saturated or unsaturated carboxylic acid having a chain length of from 15 to 36 C atoms and a linear or branched, saturated, partially saturated or unsaturated alkanol having a chain length of from 15 to 36 C atoms. Examples of ester waxes according to the invention are cetyl palmitate and myristyl palmitate.

Further waxes according to the invention are triglycerides which have been hardened by hydrogenation of double bonds of unsaturated fatty acid radicals. Examples of said waxes according to the invention are hydrogenated castor oil, hydrogenated palm oil and hydrogenated coconut oil.

The cosmetic preparation according to the invention comprises one or more waxes, preferably two waxes, one of the waxes having a melting temperature between 40° C. and 50° C. at 1013 hPa and the other wax having a melting temperature of more than 50° C. at 1013 hPa. An advantage of the above selection is that the resulting cosmetic preparations have dimensional stability and/or an improved firmness and are, at the same time, experienced as smooth by the consumer.

A wax combination preferred according to the invention comprises hydrogenated castor oil and cetyl palmitate.

It is particularly preferred when, besides hydrogenated castor oil and cetyl palmitate, no further waxes are present in the cosmetic preparations according to the invention.

Advantageously, the total proportion of the one or more waxes which melt from a temperature of 40° C. at 1013 hPa without decomposition in the cosmetic preparations is from 8% by weight to 15% by weight, preferably from 10% by weight to 13% by weight, especially preferably from 11% by weight to 12% by weight.

Furthermore, the cosmetic preparations according to the invention contain one or more saturated and/or unsaturated linear carboxylic acids having a chain length of from 10 to 20 C atoms. Said carboxylic acids are, in the context of the invention, certainly not referred to as emulsifer, oil or wax.

The carboxylic acids according to the invention are, inter alia, selected from the group capric acid, lauric acid, myristic acid, palmitic acid, stearic acid, oleic acid and arachidic acid.

Preference is given to using mixtures of two or more carboxylic acids according to the invention.

An example of such a mixture particularly preferred according to the invention comprises myristic acid, palmitic acid, stearic acid, arachidic acid and oleic acid, The above carboxylic acid mixture is commercially available under the trade name Omyacid 48 (cosmed grade) from Omya Peralta.

It is advantageous according to the invention when the one or more saturated and/or unsaturated linear carboxylic acids having a chain length of from 10 to 20 C atoms are present in the cosmetic preparation according to the invention in a total proportion of from 2.0% by weight to 5.0% by weight and especially preferably in a total proportion of from 2.5% by weight to 4.5% by weight.

The classic mechanism of action of active ingredients with antiperspirant action is based predominantly on blockage of the sweat duct of the eccrine sweat gland, including through desquamated corneocytes of the cornified secretory duct sticking together.

The cosmetic preparations according to the invention contain one or more active ingredients with antiperspirant action.

In one advantageous embodiment of the invention, one or more aluminum salts and/or aluminum/zirconium salts are present as active ingredients with antiperspirant action. These are advantageously present in the cosmetic preparation according to the invention in a proportion of from 12.5% by weight to 25% by weight, especially preferably in a proportion of from 15% by weight to 20% by weight.

The specified percentages by weight that are listed are based on the active content of the active ingredients, i.e., on anhydrous complexes in the case of the aluminum compounds and on anhydrous and buffer-free complexes in the case of the aluminum/zirconium compounds. Glycine is usually used as buffer of the aluminum/zirconium compounds.

Aluminum salts according to the invention are:
Aluminum salts such as aluminum chloride AlCl₃, aluminum sulfate Al₂(SO₄)₃
Aluminum chlorides of the empirical molecular formula [Al₂(OH)ₘClₙ], where m+n=6
Aluminum chlorohydrate [Al₂(OH)₅Cl]×H₂O (ACH)
   Standard Al complexes: Locron P (Clariant), Locron L (Clariant), Micro-Dry (Reheis), ACH-331 (Summit), Aloxicoll PF 40 (Giulini).
   Activated Al complexes: Reach 501 (Reheis), AACH-324 (Summit), AACH-7171 (Summit), Aloxicoll P (Giulini), Aloxicoll SD100
Aluminum sesquichlorohydrate [Al₂(OH)_{4.5}Cl_{1.5}]×H₂O
   Standard Al complexes: Aluminum Sesquichlorohydrate (Reheis), AACH-308 (Summit)
   Activated Al complexes: Reach 301 (Reheis)
Aluminum dichlorohydrate [Al₂(OH)₄Cl₂]'H₂O

Aluminum/zirconium salts according to the invention are:
Aluminum/zirconium trichlorohydrex glycine [Al₄Zr(OH)₁₃Cl₃]×H₂O x Gly
   Standard Al/Zr complexes: Rezal 33GP (Reheis), AZG-7164 (Summit), Zirkonal P3G (Giulini)
   Activated Al/Zr complexes: Reach AZZ 902 (Reheis), AAZG-7160 (Summit), Zirkonal AP3G (Giulini)
Aluminum/zirconium tetrachlorohydrex glycine [Al₄Zr(OH)₁₂Cl₄]×H₂O×Gly
   Standard Al/Zr complexes: Rezal 36G (Reheis), AZG-368 (Summit), Zirkonal L435G (Giulini)
   Activated Al/Zr complexes: Reach 908 (Reheis), AAZG-7167 (Summit), Zirkonal AP4G (Giulini)
Aluminum/zirconium pentachlorohydrex glycine [Al₈Zr(OH)₂₃Cl₅]×H₂O×Gly
Aluminum/zirconium octachlorohydrex glycine [Al₈Zr(OH)₂₀Cl₈]×H₂O×Gly

The use of aluminum chlorohydrate (ACH) is preferred according to the invention. In this connection, it is advantageous when no further aluminum-containing active ingredients with antiperspirant action are present besides ACH and when ACH is incorporated from aqueous solution into the cosmetic preparations of the invention.

In a further advantageous embodiment of the invention, piperidinium salts of the formula (I)

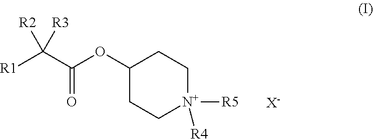

are present as one or more active ingredients with antiperspirant action, where radicals R₁ to R₅ and X⁻ have the following meanings:

R₄ and R₅ are independently of one another H or C₁-C₆-alkyl;

R₁ and R₃ are independently of one another optionally substituted C₃-C₈-cycloalkyl, optionally substituted phenyl or optionally substituted, saturated or unsaturated C₁-C₆-alkyl;

R₂ is H, OH or OR, where R is C₁-C₇-alkyl (linear, branched, cyclic, saturated or unsaturated, such as, for example, methyl, ethyl, n-propyl, isopropyl, allyl, propargyl, n-butyl, cyclopentyl and cyclohexyl); and

X⁻ is a cosmetically or dermatologically compatible anion.

In this connection, preferred piperidinium salts are selected from the 4-[(2-cyclopentyl-2-hydroxy-2-phenylacetyl)oxy]-1,1-dimethylpiperidinium salts and the 4-[(2-cyclohexyl-2-hydroxy-2-phenylacetyl)oxy]-1,1-dimethylpiperidinium salts. 4-[(2-cyclopentyl-2-hydroxy-2-phenylacetyl)oxy]-1,1-dimethylpiperidinium bromide is especially preferred.

Advantageously, the total proportion of piperidinium salts in the cosmetic preparation according to the invention is from 0.01% by weight to 15% by weight, preferably from 0.05% by weight to 10% by weight.

According to the invention, the cosmetic preparation according to the invention contains water. Advantageously, the proportion of water in the cosmetic preparation according to the invention is at least 25% by weight, preferably at least 30% by weight and especially preferably 32% by weight.

It has been found that, surprisingly, an improved feeling of freshness is advantageously perceived by the user as the proportion of water increases, especially from a proportion of water of at least 32% by weight. Furthermore, the of proportion of oily residues following application on the skin decreases as the proportion of water increases, and this is experienced by the consumer as advantageous and pleasant.

Sweat odor largely consists of branched-chain fatty acids which are released from odorless sweat by bacterial enzymes. Accordingly, it is advantageous when the cosmetic preparation additionally contains one or more further active ingredients which counteract the release of the branched-chain fatty acids by reducing the growth and multiplication of bacteria, primarily Staphylococcus epidermidis and Corynebacterium jeikeium. According to the invention, said active ingredients can be selected from the group consisting of polyquaternium-16, polyquaternium-6 and octenidine.

In the context of the invention, cosmetic preparations are referred to as dimensionally stable when they, removed from the mold in a commercially available shape and size defined via a dispenser as known from the product Nivea protect&care antiperspirant (43 g of substance, Mintel No. 3570701), do not change their outer shape within 24 h under the following conditions and with subsequent acclimatization at room temperature (21° C., 50% relative air humidity):
1. Storage at -10° C. and 50% relative air humidity for 7 days
2. Storage at 50° C. and 50% relative air humidity for 6 days
3. Storage in an alternating-temperature cabinet for 6 days, where the temperature is alternated every 12 h between −10° C. and +40° C. at 50% relative air humidity and the cooling or heating time in each case is from 5 minutes to 2 hours.

The final examination is done with the naked eye. Any noticeable change compared to the original shape is to be considered to be dimensional instability. An interim melting, breaking or cracking of the cosmetic preparation are also to be considered as a change to the outer shape.

The cosmetic preparations according to the invention are further characterized in that they are solid. In the context of the present invention, a cosmetic preparation is to be referred to as solid when it has a penetration force value in the range from 300 gram-force to 600 gram-force, preferably from 400 gram-force to 500 gram-force.

According to the invention, the penetration force value of cosmetic preparations is determined as follows: Using a TA-XT2i texture analyzer from Stable Micro Systems (Godalming, Surrey, UK), a stainless-steel cone (45°, model TA 15) is driven into the cosmetic preparation at a feed rate of 2 mm/second in a perpendicular (axial) manner up to a penetration depth of 5.000 mm. At the same time, it is ascertained what maximum force must be expended in order to allow the stainless-steel cone to penetrate into the cosmetic preparation to a depth of 5.000 mm at the specified feed rate. The maximum force expended corresponds to the penetration force value. The penetration force value is specified in gram-force. A penetration force value of 1 gram-force means that the force exerted by a mass of 1 g must be used in order to drive the stainless-steel cone into the cosmetic preparation in line with the measurement parameters. The greater the penetration force value, the more solid the preparation. According to the invention, the penetration force value is measured at an ambient temperature of from 20° C. to 27° C. and from 25% to 60% relative air humidity. A measurement is only valid when the cosmetic preparation does not break. The described measurement method has also already been described in WO 2006012971 A1.

Besides their antiperspirant action, a characteristic of deodorants is the perfuming. According to the invention, the described preparations can be present in a fragrance-neutral state (non-perfumed) or preferably in a perfumed state.

For the present invention, perfume substances are selected from the group cis-3-hexenol (CAS 928-96-1), coumarin (CAS 91-64-5), butylphenyl methylpropional (80-54-6), orange terpenes (CAS 8028-48-6), beta-ionone (CAS 8013-90-9), orange oil (CAS 8008-57-9), terpineol (CAS 8000-41-7), linalool (CAS 78-70-6), tetrahydrolinalool (CAS 78-69-3), triethyl citrate (CAS 77-93-0), allyl amyl glycolate (CAS 67634-00-8), 2-isobutyl-4-hydroxy-4-methyltetrahydropyran (CAS 63500-71-0), hexyl salicylate (CAS 6259-76-3), phenylethyl alcohol (CAS 60-12-8), 3-methyl-5-phenyl-1-pentanol (CAS 55066-48-3), 2-acetonapthone-1,2,3,4,5,6,7,8-octahydro-2,3,8,8-tetramethyl (CAS 54464-57-2), methyl cedryl ketone (CAS 32388-55-9), methyl dihydrojasmonate (CAS 24851-98-7), 2,6-dimethyl-7-octen-2-ol (CAS 18479-58-8), benzyl acetate (CAS 140-11-4), methyl-alpha-ionone (CAS 1335-46-2), heliotropin (piperonal, CAS 120-57-0), benzyl salicylate (CAS 118-58-1), linalyl acetate (CAS 115-95-7), geraniol (CAS 106-24-1), citronellol (CAS 106-22-9), omega-pentadecalactone (CAS 106-02-5), ethylene brassylate (105-95-3), ethyl linalool (CAS 10339-55-6) and hexyl cinnamal (CAS 101-86-0).

The cosmetic preparations according to the invention can further contain cosmetic excipients and active ingredients, as are usually used in such preparations, for example active ingredients, preservatives, preservation aids, lipids, antifoams, dyes and color pigments, thickeners, moisturizing and/or humectant substances, or other customary constituents of a cosmetic or dermatological formulation such as polymers, foam stabilizers, organic solvents or silicone derivatives, provided that the addition does not impair the required properties with respect to stability or is ruled out.

In the examples listed below, the present invention is described by way of example without there being any intention to limit the invention, the scope of application of which is apparent from the entire description and the claims, to the embodiments stated in the examples.

The specified numbers in example formulations 1 to 7 are percentages by weight based on the total mass of the preparation, unless otherwise specified. The specified proportions refer to the proportion by weight of the particular INCI substance.

| INCI | Ex. 1 | Ex. 2 | Ex. 3 | Ex. 4 | Ex. 5 | Ex. 6 | Ex. 7 |
|---|---|---|---|---|---|---|---|
| Aluminum | 15.0 | 15.0 | 15.0 | 20.0 | 15.0 | 17.5 | |
| chlorohydrate | | | | | | | |
| 4-[(2-cyclopentyl-2- | | | | | | | 1.0 |
| hydroxy-2- | | | | | | | |
| phenylacetyl)oxy]-1,1- | | | | | | | |
| dimethylpiperidinium | | | | | | | |
| bromide | | | | | | | |
| Cetyl palmitate | 6.5 | 6.0 | 6.0 | 5.5 | 7.0 | 5.8 | 5.8 |
| Hydrogenated castor oil | 5.5 | 6.0 | 6.0 | 6.0 | 5.0 | 5.7 | 5.7 |
| PPG-14 butyl ether | 6.4 | 5.0 | 5.2 | 7.5 | 7.0 | 5.8 | 5.8 |
| PPG-15 stearyl ether | 10.0 | 10.0 | 10.2 | 8.0 | 11.0 | 9.5 | 9.5 |
| C12-15 alkyl benzoate | 5.3 | 5.1 | 5.2 | 7.5 | 7.0 | 7.0 | 7.0 |
| Steareth-21 | 0.5 | 0.5 | 0.5 | 0.5 | 0.6 | 0.5 | 0.5 |
| Steareth-2 | 0.5 | 0.5 | 0.5 | 0.5 | 0.5 | 0.5 | 0.5 |
| Oleth-20 | 1.0 | 1.0 | 1.0 | 1.0 | 1.1 | 1.0 | 1.0 |
| ****Palmitic acid + stearic | 4.0 | 3.8 | 3.8 | 3.5 | 3.5 | 4.0 | 4.0 |
| acid + myristic acid + | | | | | | | |
| arachidic acid + oleic | | | | | | | |
| acid | | | | | | | |
| Propylene glycol | 1.5 | 1.0 | 2.5 | 2.5 | 2.0 | 2.0 | 2.0 |
| Cetearyl alcohol | | 2.5 | 2.5 | | 3 | 2.6 | 2.6 |
| Cetyl alcohol | 1.2 | | | 0.8 | | | |
| Stearyl alcohol | 1.6 | | | 1.7 | | | |
| C20-40 alkyl stearate | | 1.0 | | | | | |
| Perfume | q.s. | q.s. | q.s. | q.s. | q.s. | q.s. | q.s. |
| Water | ad 100 | ad 100 | ad 100 | ad 100 | ad 100 | ad 100 | ad 100 |

****Raw material mixture composed of the following carboxylic acids: Palmitic acid 48.5% Stearic acid 47% Myristic acid 2.5% Arachidic acid 1.5% Oleic acid 0.5%

1.-28. (canceled)

29. A cosmetic preparation, wherein the preparation is in the form of an O/W emulsion and comprises
(a) from 0.1% to 2.5% by weight of one or more alkane-1,2-diols with an alkyl chain having from 3 to 5 C atoms,
(b) two or more emulsifiers which comprise at least one O/W emulsifier having an HLB value greater than 8 and at least one W/O emulsifier having an HLB value up to a maximum of 8,
(c) one or more fatty alcohols selected from aliphatic, linear, primary alkanols having a chain length of from 6 to 22 C atoms,
(d) one or more oils which are liquid at 20° C. and 1013 hPa,
(e) one or more waxes which melt from a temperature of 40° C. at 1013 hPa without decomposition,
(f) one or more saturated and/or unsaturated linear carboxylic acids having a chain length of from 10 to 20 C atoms,
(g) one or more active ingredients with antiperspirant action, and
(h) water,
based in each case on a total weight of the preparation,
and wherein the preparation exhibits a penetration force value of from 300 gram-force to 600 gram-force.

30. The preparation of claim 29, wherein (a) is present in a total proportion of from 0.2% to 2% by weight, based on a total weight of the preparation.

31. The preparation of claim 29, wherein (a) comprises propane-1,2-diol.

32. The preparation of claim 29, wherein a total proportion of (b) is from 1.5% to 5% by weight based on a total weight of the preparation.

33. The preparation of claim 29, wherein (b) comprises two nonionic O/W emulsifiers and one nonionic W/O emulsifier.

34. The preparation of claim 29, wherein the emulsifiers of (b) are individually present in a proportion of from 0.5% to 1% by weight, based on the total weight of the preparation.

35. The preparation of claim 29, wherein (b) comprises steareth-21 and oleth-20 as nonionic O/W emulsifiers.

36. The preparation of claim 29, wherein (b) comprises steareth-2 as nonionic W/O emulsifier.

37. The preparation of claim 29, wherein (c) is present in a total proportion of from 0.5% to 5% by weight, based on a total weight of the preparation.

38. The preparation of claim 29, wherein (c) comprises one or both of cetyl alcohol and stearyl alcohol.

39. The preparation of claim 29, wherein (d) is present in a proportion of from 18% to 27% by weight, based on a total weight of the preparation.

40. The preparation of claim 29, wherein (d) comprises two ether oils and one ester oil.

41. The preparation of claim 40, wherein the ether oils are PPG-14 butyl ether and PPG-15 stearyl ether.

42. The preparation of claim 40, wherein the ester oil is C12-15 alkyl benzoate.

43. The preparation of claim 40, wherein a weight ratio of the ether oils to the ester oil is from 4:1 to 1:1.

44. The preparation of claim 29, wherein a total proportion of (e) is from 8% to 15% by weight, based on a total weight of the preparation.

45. The preparation of claim 29, wherein (e) comprises hydrogenated castor oil and cetyl palmitate.

46. The preparation of claim 29, wherein (f) is present in a total proportion of from 2.0% to 5.0% by weight, based on a total weight of the preparation.

47. The preparation of claim 29, wherein (g) comprises one or more aluminum and/or aluminum/zirconium salts.

48. A method of reducing or inhibiting perspiration, wherein the method comprises applying to skin a quantity of the cosmetic preparation of claim 29 which is effective against perspiration.
